(19) United States
(12) Patent Application Publication
(10) Pub. No.: US 2009/0312277 A1
(43) Pub. Date: Dec. 17, 2009

(54) Compositions And Methods For Reversing Or Preventing Resistance Of A Cancer Cell To A Cytotoxic Agent

(76) Inventor: Abdelhadi Rebbaa, Guilderland, NY (US)

Correspondence Address: BRINKS HOFER GILSON & LIONE, P.O. BOX 10395, CHICAGO, IL, 60610, US

(21) Appl. No.: 11/667,905

(22) PCT Filed: Nov. 18, 2005
(86) PCT No.: PCT/US2005/041869

§ 371 Date: Aug. 26, 2008

Related U.S. Application Data
(60) Provisional application No. 60/629,807, filed on Nov. 19, 2004.

(51) Int. Cl. A61K 31/704; C12N 5/08; A61K 31/7105; A61K 31/713; A61P 35/00

(52) U.S. Cl. 514/34; 435/377; 514/44 A

(57) ABSTRACT

The present invention provides a composition and method for increasing sensitivity of a cancer cell and to reverse or prevent resistance to a cytotoxic agent. The composition includes an inhibiting agent and, optionally, a cytotoxic agent. The inhibiting agent is present in a sub-lethal dose with respect to the cancer cell in the absence of the cytotoxic agent.

### PRIORITY INFORMATION

This application claims priority to U.S. Provisional Application Ser. No. 60/629,807, filed Nov. 19, 2004, which is incorporated by reference in its entirety in all jurisdictions wherein such incorporation is acceptable.

### BACKGROUND

1. Technical Field

The present invention relates to the field of oncology. In particular, the present invention relates to pharmaceutical compositions and methods for reversing and preventing resistance of cancer cells to cytotoxic agents.

2. Background Information

Cancer cells, like any cells, can become resistant to the cytotoxic agents used today in chemotherapy. Such gained resistance is the main cause of patient relapse after an otherwise successful round of chemotherapy. The mechanism by which cancer cells evade the effects of a cytotoxic agent is the subject of much research. In fact, resistant cancer cells have been shown to use a variety of strategies to overcome the chemotherapy. One class of drug-resistant cancer cells have been shown to have altered membrane transport and/or altered cellular enzymes that serve to exclude, sequester, or neutralize cytotoxic agents. This results in the cytotoxic agent having no or reduced effect on the targeted cancer cells. Other mechanisms by which cancer cells have become resistant to cytotoxic agents include over-expression of the drug efflux transporter P-glycoprotein and detoxifying enzyme glutathione-S-transferase, and DNA damage repair enzyme 6-methyl-transferase, none of which have thus far been successfully used to improve chemotherapeutic outcome.

Previous attempts to reverse resistance of cancer cells to chemotherapeutic drugs have had limited success. For example, inhibitors of P-glycoprotein, such as cyclosporine A and verapamil, were able to reverse drug resistance in vitro, but failed to do so in vivo (for a review, see Thomas et al., 2003, Cancer Control, 10 (2): 159-165)

Therefore, there is a need for a composition and method for reversing, preventing or retarding the rate of resistance of cancer cells to chemotherapeutic drugs, including, for example, enhancing the sensitivity of cancer cells to cytotoxic agents such that a lower dose of the chemotherapeutic drug may be administered.

### BRIEF SUMMARY

In one embodiment, the present invention provides a composition for increasing sensitivity of a cancer cell to a cytotoxic agent. The composition includes an inhibiting agent and the cytotoxic agent. The inhibiting agent is present in sub-cytotoxic concentration, with respect to the cancer cell, in the absence of the cytotoxic agent.

In another embodiment, the present invention provides a composition for increasing sensitivity of a cancer cell to a chemotherapeutic agent. The composition includes an inhibiting agent and the chemotherapeutic agent. The inhibiting agent is a nucleic acid present in an amount sufficient to down regulate expression of the target gene.

In another embodiment, the present invention is a composition for increasing sensitivity of a cancer cell to a chemotherapeutic agent. The composition includes a cathepsin inhibitor and a chemotherapeutic agent. The cathepsin inhibitor is present in a sub-cytotoxic concentration, with respect to the cancer cell, in the absence of the chemotherapeutic agent.

In another embodiment, the present invention provides a composition for increasing sensitivity of a cancer cell to a chemotherapeutic agent. The composition includes a cathepsin inhibitor and the chemotherapeutic agent. The cathepsin inhibitor is a nucleic acid present in an amount sufficient to down regulate expression of the target gene.

Another embodiment of the present invention provides a method for increasing sensitivity of a cancer cell to a chemotherapeutic agent. The method includes contacting a cancer cell with a cathepsin inhibitor and a chemotherapeutic agent. The cathepsin inhibitor is present in a sub-cytotoxic concentration, with respect to the cancer cell, in the absence of the chemotherapeutic agent.

Another embodiment of the present invention provides a method for increasing sensitivity of a cancer cell to a chemotherapeutic agent. The method includes contacting a cancer cell with a cathepsin inhibitor and a chemotherapeutic agent. The cathepsin inhibitor is a nucleic acid present in an amount sufficient to down regulate expression of the target gene. The nucleic acid is an siRNA, an shRNA, an antisense, or an antisense RNA that, after entry into the cell, inhibits expression of the cathepsin gene.

In another embodiment, the invention is a method for increasing sensitivity of a cancer cell to a chemotherapeutic agent. The method includes contacting the cancer cell with a composition of a cathepsin inhibitor and a chemotherapeutic agent. The effective dose of the chemotherapeutic agent in the composition is less than the effective dose of the chemotherapeutic agent administered in the absence of the cathepsin inhibitor.

In another embodiment, a method for increasing sensitivity of a cancer cell to a cytotoxic agent is provided. The method includes contacting the cancer cell with a zinc finger protein that specifically inhibits expression of a cathepsin gene. The zinc finger binds to at least about 12 contiguous nucleotides of SEQ ID NO: 7, SEQ ID NO: 8 or SEQ ID NO: 9.

In another embodiment, the invention provides an isolated nucleic acid that includes a sequence that is SEQ ID NO: 1, or is a sequence that is about 80% identical to SEQ ID NO: 1, or a sequence that is complementary to SEQ ID NO: 1 or is complementary to a sequence that is 80% identical to SEQ ID NO: 1.

Another embodiment of the present invention provides a double stranded RNA that has at least 80% identity with SEQ ID NO: 1.

A method of preventing resistance of a cancer cell to a chemotherapeutic agent is also provided. The method includes contacting a cancer cell with a cathepsin inhibitor and a chemotherapeutic agent. The cathepsin inhibitor is present in a sub-cytotoxic concentration, with respect to the cancer cell, in the absence of the chemotherapeutic agent.

A composition for increasing sensitivity of a cancer cell to a chemotherapeutic agent is provided in another embodiment. The composition includes at least one of a cathepsin L inhibitor, a cathepsin K inhibitor, and a cathepsin S inhibitor. The inhibitor is present in a concentration that is less than 100 μM.

In another embodiment, a method of increasing sensitivity of a cancer cell to a chemotherapeutic agent is provided. The method includes contacting the cancer cell with at least one of a cathepsin L inhibitor, a cathepsin K inhibitor, and a cathepsin S inhibitor. The inhibitor is present in a concentration that is less than 100 μM.

In another embodiment, an isolated nucleic acid comprising SEQ ID NO: 1, a sequence that is at least about 80% identical to SEQ ID NO: 1, a complement to SEQ ID NO: 1, or a complement to the sequence that is at least about 80% identical to SEQ ID NO: 1; provided that the isolated nucleic acid sequence is not SEQ ID NO: 7, SEQ ID NO: 8, or SEQ ID NO: 9.

### BRIEF DESCRIPTION OF THE SEQUENCES

SEQ ID NO:1 is a sequence complementary to a segment of the coding region of the human cathepsin L gene.

SEQ ID NO: 2 is a segment of base pairs 91-111 of the human cathepsin L cDNA.

SEQ ID NO: 3 is an antisense segment of cathepsin L gene.

SEQ ID NO: 4 is a T7 promoter.

SEQ ID NO: 5 is a 5′ or 3′ primer.

SEQ ID NO: 6 is a forward primer for a p21/WAF1 gene.

SEQ ID NO: 7 is the full-length human cathepsin L cDNA.

SEQ ID NO: 8 is the full-length human cathepsin K cDNA.

SEQ ID NO: 9 is the full-length human cathepsin S cDNA.

SEQ ID NO: 10 is a reverse primer for a p21/WAF1 gene.

### DETAILED DESCRIPTION OF THE DRAWINGS AND THE PRESENTLY PREFERRED EMBODIMENTS

The present invention relates to a composition and method of use for the composition for the purpose of preventing, or reducing the resistance that cancer cells commonly develop in response to chemotherapeutic treatment. Such resistance can develop in a cell line studied in vitro where, for example, a cytotoxic agent is placed in contact with the cells starting at a low concentration, gradually increasing the concentration over a period of many cell divisions, and resulting in a resistant derivative cell line. The present invention is useful for treating cells that have resistance to a cytotoxic agent, whether developed in the laboratory or derived from a cancer patient whose response to a chemotherapeutic drug that included the cytotoxic agent had become refractory. When contacted with the inventive composition, these cells exhibit lesser or no continuing resistance (increased sensitivity) to the cytotoxic agent. Accordingly, the present invention has practical utility even in the context of the laboratory setting just mentioned, and exemplified below, to identify effective chemotherapeutic regimes in general, and suitable such regimes for a particular patient where the laboratory study centers on cancer cells derived from that patient.

Such chemotherapeutic regimes for a patient involve localized or systemic administration to the patient of a cytotoxic agent that, preferably, is specific to the particular cancer cells found in the patient. However, such specificity is rare, and common cytotoxic agents used in the context of chemotherapeutic treatment of cancer usually have a detrimental impact on any actively dividing cell in the patient's body. Accordingly, merely applying increasing doses of a chemotherapeutic drug in the face of mounting resistance by the cancer cells to the cytotoxic agent in the drug is generally not an option. The inventive composition preferably functions to reverse resistance to a chemotherapeutic drug by a cancer cell. Further, this composition more preferably functions to prevent resistance from occurring. In other words, this composition functions to increase the sensitivity of a cancer cell to a cytotoxic agent, thereby allowing a subject in need of chemotherapy to require lesser administrations of a chemotherapeutic drug to check or reverse the uncontrolled growth of the cancer cells themselves.

The following definitions are presented for the purpose of facilitating understanding by the reader of the present invention; these definitions are not intended to limit the scope of the claimed invention.

Definitions.

The term “cathepsin” refers to any of several lysosomal enzymes that degrade protein and are commonly involved in the breakdown of all or part of a cell. Cathepsins have been described as having L, S, K, and B varieties; of these, the L, S, and K varieties have been shown to be the gene products of a multi-gene family where each is encoded by separate but related genes; referred to herein as the “cathepsin L family.” Cathepsins have been implicated in antigen presentation and osteoporosis (see Turk et al., 2001, EMBO J, 20 (2): 4629-4633). Their inhibition hitherto has not been known to have a role in reversing resistance of a cancer cell to a cytotoxic agent.

The phrase “cathepsin inhibitor” refers to a small molecule that inhibits the activity of a cathepsin.

The phrase “drug resistance” or “drug resistant” refers to the decreased ability of a cell to respond to a given pharmaceutical composition, which is referred to herein as a drug also. More particularly, the resistance of the cell is with respect to the cytotoxic agent included in the drug.

The term “expression” with respect to a gene refers to the use of that gene sequence for generating the there-encoded gene product, which can be an RNA or a protein, as appropriate. As used herein, expression of an antisense molecule refers to transcription of the gene only and, expression of a protein refers to both transcription of the gene to form the MRNA and translation of the mRNA to form the protein.

The term “gene” refers to a nucleic acid that includes at least the coding sequence for a gene product of interest, i.e., the DNA that encodes the gene product, which itself can be an RNA or a protein. More preferably, particularly when referred to as a full-length genomic sequence, a gene includes any or all regulatory elements, such as a promoter or enhancer, and untranslated regions, such as a 3′UTR, a 5′UTR, or intron(s), as appropriate to the gene of interest. A gene can be genomic or, especially where only the coding region is of interest, a cDNA sequence.

The term “target gene” or “target nucleic acid,” used interchangeably herein, refers to nucleic acids coding a cathepsin and includes DNA encoding a cathepsin, RNA (including pre-mRNA and mRNA) transcribed from such DNA, and cDNA derived from such RNA The specific hybridization with an oligomeric compound with its target nucleic acid interferes with the normal function of the nucleic acid. The function of DNA to be interfered with includes transcription. The function of RNA to be interfered with includes all vital functions, including, translation.

The term “gene silencing” refers to the suppression of gene expression, whether expression of a transgene, heterologous gene or endogenous gene. Gene silencing may be mediated through processes that affect transcription, translational or post-translational mechanisms. Post-transcriptional gene silencing may occur when ds RNA or siRNA are introduced into a cell and subsequently initiate the degradation of the mRNA of a gene of interest in a sequence-specific manner via “RNA interference” or “RNAi”. (for a review, see Brantl, 2002, Biochim. Biophys. Acta, 1575(1-3): 15-25). Gene silencing may also be mediated by various approaches using antisense RNA. In this approach, an RNA that includes an antisense region in its sequence with respect to the gene that is desirably silenced or suppressed, interacts with the gene and disrupts transcription thereof, the antisense region of the RNA can be the complement of a translated. Gene silencing may be allele-specific, wherein specific silencing of one allele of a gene occurs (allele being alternative forms of a gene, of which humans generally have two, one from maternal and one from paternal contribution).

The term “zinc finger” or “zinc finger proteins”, used interchangeably herein, refer to zinc containing proteins that bind to DNA in a sequence-specific manner and may be used to up or down regulate expression of a target gene.

The term “identity,” in the context of two or more nucleic acid sequences, refers to two or more sequences or subsequences that are the same or have a specified percentage of nucleotides that are the same (such as, at least about 80%, preferably about 85%, about 90%, about 95%, about 96%, about 97%, about 98%, about 99% identity over a specified region), when compared and aligned for maximum correspondence over a comparison window, or designated region as measured using a sequence comparison algorithm or by manual alignment and visual inspection. This definition, when the context indicates, also refers analogously to the complement of a sequence, such as an RNA nucleotide complementary to a DNA nucleotide. Preferably, substantial identity exists over a region that is at least about 25 nucleotides in length.

The phrase “increasing sensitivity of a cancer cell to a cytotoxic agent” refers to making a cancer cell susceptible to the detrimental effects of a cytotoxic agent to which it was previously resistant or, alternatively, making a cancer cell susceptible to a cytotoxic agent at a lower dose or concentration thereof than was the case prior to contacting the cell with the inventive composition.

The phrase “neutral base changes” refers to one or more changes in the sequence of a nucleic acid such that sufficient binding to a target sequence may occur and cause the desired effect.

The term “small interfering RNA” or “short interfering RNA” or “siRNA”, each of which are used interchangeably herein, refers to a nucleic acid that forms a double-stranded RNA, which double-stranded RNA has the ability to reduce or inhibit expression of a gene or target gene when the siRNA is expressed in the same cell as the gene or target gene. “siRNA” thus refers to the double-stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double-stranded siRNA. The sequence of the siRNA can correspond to the full-length target gene, or a subsequence thereof siRNA is “targeted” to a gene in that the nucleotide sequence of the duplex portion of the siRNA is substantially complementary to a nucleotide sequence of the targeted gene. The siRNA sequence duplex needs to be of sufficient length to bring the siRNA and target RNA together through complementary base-pairing interactions. The siRNA of the invention may be of varying lengths. The length of the siRNA is preferably greater than or equal to 10 nucleotides and of sufficient length to stably interact with the target RNA; specifically, 10-30 nucleotides; more specifically, any integer between 10 and 30 nucleotides, such as 10, 11, 12, 13, 14, 15, 16, 17, 18, 19, 20, 21, 22, 23, 24, 25, 26, 27, 28, 29, and 30. By “sufficient length” it is meant a nucleotide of greater than or equal to 10 nucleotides that is of a length great enough to provide the intended function under the expected condition. The term “stably interact” refers to interaction of the siRNA with target nucleic acid (e.g., by forming hydrogen bonds with complementary nucleotides in the target under physiological conditions).

The siRNA may be encoded by a nucleic acid sequence, and the nucleic acid sequence can also include a promoter. The nucleic acid can also include a polyadenylation signal. In some embodiments, the polyadenylation signal is a synthetic minimal polyadenylation signal. The RNA duplex of the siRNA may be constructed in vitro using synthetic oligonucleotides.

The term “antisense” refers to a nucleic acid sequence that is complementary to a DNA sequence or a sequence that is processed into mRNA and translated. Antisense need not be 100% complementary to its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of antisense compounds to non-target sequences.

The phrase “small molecule” means a molecule having amino acid analogs or small peptides that have been chemically synthesized. Alternatively, the small molecule may be a small synthetic compound containing no amino acids. The molecular weight of the small molecule should not be greater than about 300. The small molecules may be synthesized using principles and procedures commonly known to practitioners of organic chemical synthesis.

The term “transfection” is used to refer to the uptake of an exogenous (i.e., foreign) nucleic acid by a mammalian cell. A cell has been “transfected” when an exogenous nucleic acid has been introduced inside the cell membrane. Transfection can be used to introduce one or more exogenous nucleic acid constructs, such as a plasmid, vector and other nucleic acid molecules, into a suitable host cell. The term refers to both stable and transient uptake of genetic material.

Practicing the present invention requires use of conventional methods well-known in the literature. The description presented here of the present invention certainly will set forth many of these methods, however to the extent that these methods are well-known, it is contemplated that the reader may augment, substitute or otherwise alter what is presented here, and nevertheless arrive at the same useful composition and method of use. Accordingly, what is set forth herein should be understood in the light of known equivalent procedures and variations thereof without viewing what is set forth as being limiting of the scope of the invention. For disclosure of many of the methods used in the context of the present invention, including many alternative methods, the reader is directed to the following exemplary publications, which should not be viewed as limiting the present invention in any manner: Molecular Cloning, A Laboratory Manual (3 Volume Set), J. Sambrook et al., published by Cold Spring Harbor Laboratory, 2001; Gene Expression Technology, Methods in Enzymology Series, Vol. 185, edited by J. N. Abelson, M. I. Simon, and D. V. Goeddel, published by Elsevier Science & Technology Books, 1990; and Culture of Animal Cells: A Manual of Basic Technique, 4th Edition, R. Ian Freshney, Wiley-Liss, 2000.

In a preferred embodiment of the present invention, a composition for increasing sensitivity of a cancer cell to a cytotoxic agent is provided. The composition preferably includes an inhibiting agent, more preferably a cathepsin inhibitor

As noted above, cathepsins L, S, K, and B are lysosomal enzymes mainly known to play a role in antigen presentation and osteoporosis. However, the present invention is predicated on the finding reported here that inhibition of cathepsin enhances cell susceptibility to the detrimental effect of cytotoxic agents.

Without being bound by any particular theory, it is believed that resistance to cytotoxic agents may be due to sequestration of the cytotoxic agent in the lysosome. Accordingly, it is believed, although not relied upon, that inhibition of cathepsin (preferably cathepsin L, K or S; more preferably cathepsin L), and subsequent lysosomal disruption, leads to translocation of the cytotoxic agent from the lysosome to a non-lysosomal location, where the cytotoxic agent causes the cell to reduce its mitotic rate, senesce, or otherwise impair its growth characteristics.

There are various known methods of inhibiting cathepsin, including the use of small molecules that specifically bind to a cathepsin. Examples of such small molecules include, without limitation, Z-Phe-Tyr-aldehyde (iCL), N-(2-Quinolyl)valyl-O-methylaspartyl-(2,6-difluorophenoxy)methyl Ketone (Q-VD; Enzyme System Products; Livermore, Calif.), Z-Phe-Tyr-(t-Bu)-diazomethylketone (Calbiochem-Novabiochem, San Diego, Calif.), Napsule-Ile-Tryp (Biomol; Plymouth Meeting, Pa.), 1,3-Bis(N-CBZ-Leu-NH)-2-propanonel,3-Di(N-carbobenzoyloxy-L-leucyl)amino acetone, Z-Phe-Leu-COCHO, BML-244, BML-248, Calpain Inhibitor II, Calpeptin, E-64c, E-64d, available from Biomol; and Cbz-Leu-NH-CH₂-CO-CH₂-NH-Leu-Cbz, Boc-Phe-NHNH-Leu-Z, H-Phe-Leu-NHNH-CO-NHNH-Leu-Z, Z-Phe-Phe-CH₂F, Z-Phe-Tyr-CHO, 1-Naphthalenesulfoneyl-Ile-Trp-CHO, Z-Phe-Tyr(otBu)-COCHO.H₂O, (each of which is available from Calbiochem-Novabiochem, San Diego, Calif.). In addition, natural inhibitors, such as cystatins, lactacystins, and serpins may be used. Inhibitors of mannose-6-phosphate may also be used.

Preferably, the cathepsin inhibitor is conjugated, i.e., linked, to a moiety that provides stability, aids delivery, or increases specificity of the inhibitor to the target. For example, any moiety that helps to make the cathepsin inhibitor liposoluble or targets lysosome tags may be used. All suitable analogs and pharmaceutically-effective derivatives of the above-named small molecules that are useful in the context of the present invention are contemplated as well. Suitable such analogs and derivatives may exhibit lesser, same, or greater ability to reverse chemotherapeutic resistance relative to the parent small molecule upon which the analog or derivative is based. Methods to demonstrate usefulness of the suitable analog or derivative small molecules are set forth at Examples 1-5, 7 and 8.

Preferably, the inhibiting agent, preferably a cathepsin inhibitor, is present in the composition in a concentration that is sub-lethal to the cancer cell. That is, when administered without a cytotoxic agent, the concentration of the inhibiting agent in the composition is not lethal to the cancer cell. Preferably, the inhibiting agent is present in a concentration that is less than 100 μM. A preferred range of the sub-lethal concentration of an inhibiting agent is from about 5 μM to about 40 μM. A more preferred range is from about 10 μM to about 20 μM, and a yet more preferred range is from about 10 μM to about 15 μM.

In certain embodiments, a cathepsin inhibitor is provided in a concentration that is lethal to the cancer cell or at a concentration or administration route that decreases tumor volume. Such embodiments may comprise concentrations of a cathepsin inhibitor that are greater than 100 μM, greater than 200 μM, greater than 300 μM, greater than 400 μM, and greater than 500 μM.

Provided herein are methods for increasing the sensitivity of a cancer cell to a cytotoxic agent or preventing resistance of a cell to a cytotoxic agent. A cathepsin inhibitor is administered in a dose effective to increase the sensitivity of a cell to cytotoxic agent or prevent resistance a cell to a cytotoxic agent. Dosages include one or more administrations of a cathepsin inhibitor. Dosages of a cathepsin inhibitor include, but are not limited to, at least 30 mg/kg, at least 60 mg/kg, at least 90 mg/kg, and at least 180 mg/kg. Dosages further include less than about 180 mg/kg, less than about 90 mg/kg, less than about 60 mg/kg, and less than about 30 mg/kg.

As demonstrated in Example 1, the cathepsin inhibitor alone is effective in reducing tumor volumes in a nude mouse model system. As further demonstrated in example 1, co-administration of the cathepsin inhibitor and the cytotoxic agent showed greater efficacy than either component alone. Thus, it is contemplated that dosing regimens having reduced concentration of the cathepsin inhibitor and/or the cytotoxic agent will be as or more effective than dosing regimen's utilizing either component at greater concentrations. Using lower concentrations of the components reduces the side effects associated with higher doses of either component.

A preferred inhibiting agent is one that inhibits cathepsin L or cathepsin S or cathepsin K. A more preferred inhibiting agent inhibits cathepsin L. A yet more preferred inhibiting agent preferentially inhibits cathepsin L with respect to cathepsin S or cathepsin K. A most preferred inhibiting agent is specific to cathepsin L and has insignificant or no inhibiting activity with respect to cathepsin S or cathepsin K or cathepsin B. In yet another embodiment of the present invention, a preferred inhibiting agent inhibits any of the cathepsin L family of cathepsins. More preferably, the preferred inhibiting agent preferentially inhibits any of the cathepsin L family members as compared to its effect on cathepsin B.

Another method of increasing the sensitivity of a cancer cell to a cytotoxic agent employs a composition that comprises an inhibiting agent, where the inhibiting agent is a nucleic acid present in an amount that is effective to inhibit expression of a target gene in the cancer cell. Preferably, the target gene encodes a cathepsin; more preferably, the cathepsin is cathepsin L, or cathepsin S, or cathepsin K; and yet more preferably, the cathepsin is cathepsin L. In reducing the quantity of the targeted gene product by means of the inhibiting agent, it naturally follows that there is less activity of that gene product in the so-affected cancer cell due to a reduction in synthesis of new cathepsin, presuming that the cathepsin present in the cell is itself limited due to inherent cellular controls on its synthesis, such as feedback inhibition, for example. Examples of such inhibiting agents include, without limitation, a nucleic acid, such as an antisense DNA, an antisense RNA, a DNA, an RNA, a dsRNA, an siRNA, an miRNA, an shRNA, and a cDNA. Preferably, the nucleic acid used in the context of the present invention is specific for inhibiting the transcription of the cathepsin gene and/or translation of the mRNA specific for cathepsin.

The composition may also include a vehicle. Such vehicles include, but are not limited to, viral vectors, plasmids, bacteriophages, cosmids, retroviruses, artificial chromosomes, liposomes, and other carrier molecules that facilitate delivery and are well-known to those in the art.

If a nucleic acid is used (including antisense and siRNA), it may be natural or “modified”. If the nucleic acid is a modified antisense, it may include, by way of non-limiting example, modified backbones or non-natural internucleoside linkages, phosphorous-containing linkages, and non-phosphorous-containing linkages, short chain alkyl or cycloalkyl internucleoside linkages, mixed heteroatom and alkyl or cycloalkyl internucleoside linkages, short chain heteroatomic or heterocyclic internucleoside linkages, and morpholino linkages. Antisense may also be chemically linked to one or more moieties or conjugates which enhance activity, cellular distribution, or cellular uptake of the oligonucleotide. U.S. patents teaching the preparation and use of such conjugates, as well as the above described modifications, include U.S. Pat. No. 6,451,538, incorporated herein by reference.

The antisense used may be complementary to DNA (antisense DNA) or to MRNA (antisense RNA). Antisense RNA is used to inhibit translation of the mRNA and therefore inhibits expression of the gene of interest. Messenger RNA is single-stranded, therefore antisense RNA that is complementary to the mRNA of interest is able to bind to the sense strand of the mRNA, forming a duplex and therefore inhibiting translation of the mRNA. Preferably, the antisense molecule will bind to at least 6 contiguous nucleotides of the target nucleic acid. Techniques for utilizing antisense RNA as a gene silencing agent are well known to those in the art.

Further, transcription factors may be used to inhibit gene expression. Transcription factors typically inhibit gene expression by binding to 12 to 15 contiguous nucleotides of a DNA sequence. In a preferred embodiment, the transcription factor utilized is a zinc finger protein that specifically inhibits expression of a cathepsin gene. In a preferred embodiment, the zinc finger binds to at least about 12 to 15 contiguous nucleotides of SEQ ID NO: 7, SEQ ID NO: 8, or SEQ ID NO: 9. Zinc fingers may be used to deliver an antisense molecule to the target DNA. Zinc fingers are proteins that bind to DNA in a sequence-specific manner. A single finger domain is about 30 amino acids in length and contains an alpha helix containing the two invariant histidine residues coordinated through zinc with the two cysteines of a single beta turn. Over 10,000 zinc finger sequences have been identified in several thousand known or putative transcription factors. Zinc finger proteins are involved in not only DNA recognition, but also in RNA binding, and protein-protein binding. Zinc fingers may be used to up-regulate or down-regulate gene expression. Zinc fingers can be readily used to up or down regulate any target gene. The use of zinc fingers is well known in the art and is exemplified in U.S. Pat. No. 6,599,692, incorporated by reference herein.

If RNA is used to inhibit cathepsin, it may be stabilized or linked to suitable moieties that provide stability to the RNA within the cell and that aid delivery of the RNA to target sites. Such moieties include methyl groups, sugars, antibodies or recognition domains thereof, and cell-penetrating peptides. In one embodiment of the present invention, gene silencing is achieved utilizing a novel siRNA, where the target gene encodes a cathepsin, and where the siRNA preferably comprises from at least about 10 to about 30 contiguous nucleotides of SEQ ID NOs: 7, 8, or 9. Preferably, the target gene encodes cathepsin L. More preferably, the siRNA comprises from at least about 15 to about 25 contiguous nucleotides of one of the above-identified sequences; yet more preferably, from about 17 to about 22; and most preferably, the siRNA comprises SEQ ID NO:1. The above sequences identified as SEQ ID NOs: 7, 8, and 9 are the full-length cDNA sequences that encode human cathepsin L, K, and S, respectively. SEQ ID NO: 1 is nucleotides 91 to 111 inclusive, i.e., UUCACCUUCCGCUACGUGUUG, derived from SEQ ID NO:7.

Generally, a target sequence on the target mRNA can be selected from a given cDNA sequence corresponding to the target mRNA, preferably beginning 50 to 100 nucleotides downstream (i.e., in the 3′ direction) from the start codon. The target sequence can, however, be located in the 5′ or 3′ untranslated regions, or in the region nearby the start codon.

siRNAs may be constructed in vitro using synthetic oligonucleotides or appropriate transcription enzymes in vivo using appropriate transcription enzymes or expression vectors. The siRNAs include a sense RNA strand and a complementary antisense RNA strand annealed together by standard Watson-Crick base-pairing interactions to form the base pairs. The sense and antisense strands of the present siRNA may be complementary single-stranded RNA molecules to form a double-stranded (ds) siRNA or a DNA polynucleotide encoding two complementary portions that may include a hairpin structure linking the complementary base pairs to form the siRNA. Preferably, the duplex regions of the siRNA formed by the ds RNA or by the DNA polypeptide include about 15-30 base pairs, more preferably 19-25 base pairs. The siRNA duplex region length may be any positive integer between 15 and 30 nucleotides.

The siRNA of the invention derived from ds RNA may include partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

One or both strands of the siRNA of the invention may include a 3′ overhang. As used herein, a “3′ overhang” refers to at least one unpaired nucleotide extending from the 3′-end of an RNA strand. Thus, in an embodiment, the siRNA may include at least one 3′ overhang from 1 to about 6 nucleotides (which includes ribonucleotides or deoxyribonucleotides) in length. The length of the overhangs can be the same or different for each strand.

The siRNA of the invention may be obtained using a number of known techniques. For example, siRNA may be chemically synthesized using appropriately protected ribonucleoside phosphoroamidites and a conventional DNA/RNA synthesizer. The siRNA may be synthesized as two separate, complementary RNA molecules, or as a single RNA molecule with two complementary regions. Commercial suppliers of synthetic RNA molecules or synthesis reagents include Dharmacon Research (Lafayette, Colo.), Pierce Chemical (Rockford, Ill.), Glen Research (Sterling, Va.), ChemGenes (Ashland, Mass.), and Cruachem (Glasgow, UK).

The siRNA of the present invention may also be expressed from a recombinant plasmid either as two separate, complementary RNA molecules, or as a single RNA molecule with two complementary regions.

Selection of vectors suitable for expressing siRNA of the invention, methods for inserting nucleic acid sequences for expressing the siRNA into the plasmid, and methods of delivering the recombinant plasmid to the cells of interest are within the skill of the art. Delivery of the recombinant nucleotides to the host cell may be confirmed by a variety of assays known to those of skill in the art. Assays include Southern and Northern blotting, RT-PCR, PCR, ELISA, and Western blotting.

Also contemplated are sequences that are at least about 80% identical thereto, more preferably 85% identical thereto, more preferably 90% identical thereto, still more preferably 95% identical thereto, and more preferably 99% identical thereto. In addition sequences that are complementary to SEQ ID NO: 1 or complementary to a sequence that is at least 80% identical to SEQ ID NO: 1 may also be used to inhibit cathepsin L. Preferably the sequence that is complementary to SEQ ID NO: 1 is at least 85% identical thereto, more preferably 90% identical thereto, still more preferably 95% identical thereto, and more preferably 99% identical thereto. Sufficient identity to SEQ ID NO: 1 is found when the analog is administered and sufficient regulation of the gene of interest is achieved or alternatively, sufficient regulation of the production of the protein of interest is achieved, to allow for successful practice of the present invention. Additionally, it is contemplated that sequences may be utilized having 16-30 base pairs, more preferably 18-30 base pairs, and still more preferably 20-30 base pairs. The sequence may contain alternate 20-mers, and neutral base changes.

Further, an isolated nucleic acid, such as DNA, RNA, or dsRNA, with a sequence that is identical to or at least about 80% identical to SEQ ID NO: 1 may be used to inhibit cathepsin gene expression or production of the protein encoded by SEQ ID NO: 1, SEQ ID NO: 7, SEQ ID NO: 8, or SEQ ID NO: 9, preferably SEQ ID NO: 1. Preferably, the isolated nucleic acid is at least 85% identical to SEQ ID NO:1, more preferably 90% identical thereto, still more preferably 95% identical thereto, and more preferably 99% identical thereto.

Delivery of the nucleic acids utilized in the present invention may be by any of a number of known methods examples of which are included below.

The chemotherapeutic agent useful for the composition of the present invention may be any known cytotoxic agent used to treat cancer. Preferably, the agent is a non-metal based agent, examples of which include doxorubicin, anthracycline, vinblastine, taxol, melphalan, mitoxantrone, etoposide, cyclophosphamide and tamoxifen.

In another embodiment of the present invention, a method of treating a subject with cancer by increasing the sensitivity of a cancer cell to a chemotherapeutic agent is provided. The method includes contacting a cancer cell or a plurality of cancer cells, with the composition described above. The subject may be a mammal, specifically a horse, dog, cat or human, most preferably, a human.

In the method of the present invention, the cathepsin inhibitors may be administered alone or in conjunction with chemotherapeutic agents. They may be administered by the same or different route of administration as the chemotherapeutic agents. Further, the cathepsin inhibitor may be administered before, during, or after administration of a chemotherapeutic agent. More than one cathepsin inhibitor may be administered at once, or in successive administrations. More than one chemotherapeutic agent may also be administered with a cathepsin inhibitor.

A composition of the present invention may be administered in any desired and effective manner: as compositions for oral ingestion, or for parenteral or other administration in any appropriate manner such as intraperitoneal, subcutaneous, intratumoral, topical, intradermal, inhalation, intrapulmonary, rectal, vaginal, sublingual, intramuscular, intravenous, intra-arterial, intrathecal, or intralymphatic. The composition of the present invention may be encapsulated or otherwise protected, against gastric or other secretions, if desired. Further, the composition may be administered via implantation of a stent, or via direct injection into a tissue or organ. Transfection and electroporation are also suitable routes of administration for compositions containing a nucleic acid.

Regardless of the route of administration selected, the composition may be formulated into pharmaceutically-acceptable dosage forms by conventional methods known to those of ordinary skill in the art (e.g., see: Remington's Pharmaceutical Sciences, Mack Publishing Co., Easton, Pa.). Pharmaceutical carriers are well known in the art (e.g., see: Remington's Pharmaceutical Sciences cited above and The National Formulary, American Pharmaceutical Association, Washington, D.C.) and include sugars (e.g., lactose, sucrose, mannitol, and sorbitol), starches, cellulose preparations, calcium phosphates (e.g., dicalcium phosphate, tricalcium phosphate and calcium hydrogenphosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly[orthoesters], and poly[anhydrides]), elastomeric matrices, liposomes, microspheres, oils (e.g., corn, germ, olive, castor, sesame, cottonseed, and groundnut), cocoa butter, waxes, paraffins, silicones, talc, silicylate, and the like.

Suitable carriers used included in the composition of the present invention should be compatible with the other ingredients of the composition. Carriers suitable for a selected dosage form and intended route of administration are well known in the art, and acceptable carriers for a chosen composition, dosage form and method of administration can be determined using ordinary skill in the art.

The composition of the present invention may, optionally, contain one or more additional agents commonly used in pharmaceutical compositions. These agents are well known in the art and include but are not limited to (1) fillers or extenders, such as starches, lactose, sucrose, glucose, mannitol, silicic acid or the like; (2) binders, such as carboxymethylcellulose, alginates, gelatin, polyvinyl pyrrolidone, hydroxypropylmethyl cellulose, sucrose, acacia or the like; (3) humectants, such as glycerol or the like; (4) disintegrating agents, such as agar-agar, calcium carbonate, potato or tapioca starch, alginic acid, certain silicates, sodium starch glycolate, cross-linked sodium carboxymethyl cellulose, sodium carbonate or the like; (5) solution retarding agents, such as paraffin or the like; (6) absorption accelerators, such as quaternary ammonium compounds or the like; (7) wetting agents, such as acetyl alcohol, glycerol monostearate or the like; (8) absorbents, such as kaolin, bentonite clay or the like; (9) lubricants, such as talc, calcium stearate, magnesium stearate, solid polyethylene glycols, sodium lauryl sulfate or the like; (10) suspending agents, such as ethoxylated isostearyl alcohols, polyoxyethylene sorbitol and sorbitan esters, microcrystalline cellulose, aluminum metahydroxide, bentonite, agar-agar, tragacanth or the like; (11) buffering agents; (12), excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, polyamide powder or the like; (13) inert diluents, such as water, other solvents or the like; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, waxes or the like; (18) opacifying agents; (19) adjuvants; (20) emulsifying and suspending agents; (21), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, fatty acid esters of sorbitan or the like; (22) propellants, such as chlorofluorohydrocarbons or the like and volatile unsubstituted hydrocarbons, such as butane, propane or the like; (23) antioxidants; (24) agents which render the formulation isotonic with the blood of the intended recipient, such as sugars, sodium chloride or the like; (25) thickening agents; (26) coating materials, such as lecithin or the like; and (27) sweetening, flavoring, coloring, perfuming and preservative agents. Each such ingredient or material should be compatible with the other ingredients of the formulation. Agents suitable for a selected dosage form and intended route of administration are well known in the art, and acceptable ingredients and materials, dosage form and method of administration may be readily determined by those of ordinary skill in the art.

A composition in accordance with the present invention that are suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations can be prepared by methods well known in the art.

In one embodiment of a method of the present invention, the effective dose of the chemotherapeutic agent in the composition is less than the effective does of the chemotherapeutic agent when administered in the absence of the cathepsin inhibitor.

The present invention also provides a method of preventing resistance of a cancer cell to a chemotherapeutic agent by administering to the cancer cell the composition described above before the cancer cell has become resistant to the chemotherapeutic agent therein.

Cathepsin activity (or suppression) may be measured in vitro using a specific fluorescent substrate such as that found in a CV-Cathepsin L Detection Kit (Biomol, Plymouth Meeting, Pa.). Cathepsin concentration may be determined by Western blot and cathepsin mRNA expression may be evaluated by Northern blot. These methods of evaluation are well known in the art.

Various forms of cancer may be treated with the above composition. Such forms include but are not limited to neuroblastoma, osteosarcoma, leukemia, breast cancer, ovarian cancer, and cancer cells derived therefrom. The present invention is useful for treatment of solid and nonsolid tumors.

While the present invention has been described with specificity in accordance with certain of its preferred embodiments, the following examples serve only to illustrate the invention and are not intended to limit the same.

### EXAMPLE 1

This example illustrates that a cathepsin L inhibitor specifically reverses resistance to doxorubicin, a cytotoxic agent, in human neuroblastoma cells, both in vitro and when administered in vivo.

Human neuroblastoma SKN-SH cells (ATCC Cat. No. HTB-11) were cultured in Dulbecco's Modified Eagles Medium (DMEM; Gibco, Grand Island, N.Y.) supplemented with 10% fetal bovine serum (FBS; Sigma-Aldrich, St. Louis, Mo.) at 37° C. in a 95% Air/5% CO₂ atmosphere. Resistant cells to doxorubicin (SKN-SH/R) were selected by stepwise exposure to drug concentrations ranging from 10⁻⁹ M through 10⁻⁶ M over a time period of three months. The resulting cell line was subjected to treatment with the doxorubicin alone or in combination with: proteosome inhibitor (Lactacystin), cathepsin B inhibitor (L-3-trans-(Propylcarbamoyl)oxirane-2-carbonyl]-L-isoleucyl-L praline), cathespin L inhibitor (Z-Phe-Tyr(t-Bu)-diazomethylketone), cathespin K inhibitor (1,3-Bis(N-CBZ-Leu-NH)-2-propanonel,3 -Di(N-carbonbenzoyloxy-L-leucyl)amino acetone), or cathespin S inhibitor (Z-Phe-Leu-COCHO.H2O), (all available from Calbiochem-Novabiochem, San Diego) at 10 μM each to determine whether they affect cell response to doxorubicin.

Cytotoxic activity of doxorubicin and cysteine protease inhibitors were quantitatively determined by a calorimetric assay utilizing 3-(4,5-dimethyl-2-thiazolyl) 2,5-diphenyl tetrazolium bromide (MTT; Sigma-Aldrich, St. Louis, Mo.). Briefly, cells were seeded at 10⁴ cells/well in 96-well plates and maintained in culture for 24 hours at 37° C. in DMEM supplemented with 10% FBS. Drugs were added to designated wells and cells were incubated for 96 hours, following which MTT (10 μL of 5 mg/ml solution) was added to each 100 μl well and incubated for 4 hours at 37° C. The cells were solubilized by incubation with 100 μl of HCl 0.5N in isopropanol for 15 hours at 37° C. The optical density of this solution was measured at 570 nm and the percentage of viable cells estimated by comparison with untreated control cells.

Results were expressed as the concentration of doxorubicin at which 50% of cells remained viable, or the IC₅₀. Neither lactacystin nor the cathepsin B affected cell response to doxorubicin. The general cysteine protease inhibitor Q-VD-OPH (Q-VD) and the cathepsin L family inhibitors in the absence or in the presence of increasing concentrations of doxorubicin (LogM), did affect cell response, as shown. In particular, the IC₅₀ shifted from about −6.75 in control cells to about −6.50, −8.25, −7.10, −6.50, −7.25, and −6.75 in cells treated with lactacystin, a cathepsin L inhibitor, Q-VD, a cathepsin B inhibitor, a cathepsin K inhibitor, and a cathepsin S inhibitor, respectively (where data is ±s.e. of three determinations). Therefore, cytotoxic sensitivity of the observed cells to doxorubicin increased in the presence of Q-VD-OPH and the cathepsin L family inhibitors.

Cytotoxicity resistance is decreased in vivo upon administration of a cathepsin inhibitor. Doxorubicin resistant human neuroblastoma cells (SKN-SH/R) cells grown into 75 cm² flasks were harvested by trypsinization and centrifuged to remove trypsin. The pellet was then reconstituted in culture medium at 10⁷ cell/ml. On day one, 100 μl (10⁶ cells) were injected to the right flank of nude mice (5 per group) and after the tumor became palpable at day 11, mice were assigned to four groups: 1) controls (treated with the vehicle DMSO), 2) Dox. 1.5 mg/kg (treated with doxorubicin alone 1.5 mg/Kg), 3) iCL 30 mg/Kg (treated with cathepsin L inhibitor alone at 30 mg/Kg), and 4) Dox 1.5 mg/Kg+iCL 30 mg/Kg (treated with the combination of doxorubicin 1.5 mg/Kg and cathepsin L inhibitor 30 mg/Kg). Intraperetoneal injections were given on day 11, day 14, and day 17. On day 27, the average tumor volume of group 1 was approximately 2000 mm³; the average tumor volume of group 2 was approximately 1600 mm³; the average tumor volume of group 3 was approximately 800 mm³; and the average tumor volume of group 4 was approximately 100 mm³.

The in vivo results indicate that, whereas doxorubicin was only marginally effective at reducing tumor growth over vehicle alone, cathepsin inhibitor caused significant reduction in tumor growth. The cathepsin inhibitor alone reduced tumor volume at day 27 by about 60% greater than the control and about 50% greater than doxorubicin. When the cathepsin inhibitor was co-administered with doxorubicin, tumor volume was reduced at day 27 by about 95% greater than the control and about 94% greater than doxorubicin alone. Thus, cathepsin inhibitor alone or in combination with a chemotherapeutic agent is effective at treating drug resistant tumors.

### EXAMPLE 2

This example sets forth an experiment to measure the activity of purified cathepsin L in the presence of various protease inhibitors.

Cathepsin L activity was measured using a commercially available kit according to the manufacturer's procedure (CV-Cathepsin L Detection Kit; Biomol, Plymouth Meeting, Pa.). Purified cathepsin L (Biomol, Plymouth Meeting, Pa.) (200 ng) was incubated with each inhibitor (10 μM) in a 96 well plate for 15 min at room temperature in 100 μl of reaction buffer (100 mM sodium acetate pH 5, 1 mM EDTA (ethylenediaminetetraacetic acid), and 4 mM dithiothreitol). The protease inhibitors tested were: lactacystin and inhibitors of cathepsin L, B, S, and K. 100 μl of a fluorogenic substrate (CV-Cathepsin L Detection Kit; Biomol, Plymouth Meeting, Pa.) were added and incubated for an additional 30 min at room temperature. Fluorescence was measured in a plate reader (Victor Multilabel Counter, Perkin Elmer) at 380 nm excitation and 40 nm emission wavelengths.

Results are indicated as negative and positive controls (no cathepsin L and cathepsin L without inhibitor, respectively). Activity is reported as arbitrary units (A.U.), ±s.e. of three determinations. Approximate results derived are: 1) negative control, 0.75e+5 AU; 2) positive control, 4e+5 AU; 3) lactacystin, 4.25e+5 AU; 4) Q-VD, 1.5e+5 AU; 5) cathepsin L inhibitor, 1e+5 AU; 6) cathepsin B inhibitor, 3.5e+5 AU; 7) cathepsin S inhibitor, 3e+5 AU; 8) cathepsin K inhibitor, 1e+5 AU. The results indicate that the cathepsin L inhibitor was the most effective and that Q-VD and the cathepsin K inhibitor were also able to reduce strongly the activity of this enzyme.

From the results set forth in Example 1 hereof, cells treated with a cathepsin L inhibitor, Q-VD or a cathepsin K inhibitor showed the greatest reduction in percentage of viable cells per concentration of doxorubicin, whereas cells treated with the other cathepsin inhibitors showed a lesser reduction (see FIG. 1). The limited effect of lactacystin and the cathepsin B inhibitor on the reversal of drug resistance may be explained by the lack of specificity toward cathepsin L family cathepsins. These findings indicate that cathepsin L family cathepsins represent a primary target in reversal of resistance to doxorubicin. Of the cathepsin L family cathepsins, cathepsin K inhibition appears useful in reversing drug resistance, but cathepsin L inhibitors as well as the general cysteine protease inhibitor Q-VD appear to be stronger inhibiting agents to reduce the enzymatic activity of cathepsin L and increase the responsiveness of cells to the cytotoxic agent, in this case, doxorubicin.

### EXAMPLE 3

This example illustrates that cathepsin L inhibition reverses drug resistance to non-anthracycline drugs in various cancer types.

The effect of cathepsin L inhibitor was tested on drug sensitive and resistant (R) cell lines corresponding to various cancer types, including the human neuroblastoma cell line SKN-SH (ATCC Cat. No. HTB-11), the murine neuroblastoma cell line Neuro2A (ATCC Cat. No. CCL-131), the osteosarcoma cells Saos2 (ATCC Cat. No. HTB-85) and the leukemia cell line HL-60 (ATCC Cat. No. CCL240). The cells were treated with cathepsin L inhibitor with or without doxorubicin as described above. Cell viability was calculated after 96 hours of incubation with the drug combination. Methods used were as described in Example 1 hereof, and results represent the mean±s.e. of six determinations.

The IC₅₀ of SKN-SH wild type cells, SKN-SH wild type plus cathepsin L inhibition, SKN-SH doxorubicin resistant cells, SKN-SH doxorubicin resistant cells plus a cathepsin L inhibitor were compared. It was found that the IC₅₀ (doxorubicin LogM) values were about −8.75, −8.70, −6.50, and −8.0 for the SKN-SH wild type cells, SKN-SH wild type plus cathepsin L inhibition, SKN-SH doxorubicin resistant cells, SKN-SH doxorubicin resistant cells plus a cathepsin L inhibitor cells, respectively. In addition, Neuro2A wild type, Neuro2A wild type plus cathepsin L inhibitor, Neuro2A doxorubicin resistant cells and Neuro2A doxorubicin resistant cells plus cathepsin L inhibitor had IC₅₀ (doxorubicin LogM) values of about −7.50, −7.50, −5.50 and −7.25, respectively. Similarly, HL-60 wild type, HL-60 wild type plus cathepsin L inhibitor, HL-60 doxorubicin resistance cells and HL-60 doxorubicin resistant plus cathepsin L inhibitor cells had IC₅₀ values of about −7.25, −6.75, −4.50, and −5.50, respectively. The IC50 Saos2 wild type, Saos2 wild type plus cathepsin L inhibition, Saos2 doxorubicin resistant and Saos2 doxorubicin resistant plus cathepsin L inhibition was about −7.0, −7.10, −5.25, and −6.25, respectively. No effect of the drug combination was noticed on doxorubicin toxicity in all of the four drug-sensitive or wild cell lines (W cells).

The present findings indicate that the cathepsin L inhibitor in combination with doxorubicin was able to enhance doxorubicin toxicity in all the drug resistant cell lines tested. Interestingly, only drug resistant cells and not their drug sensitive counterparts were affected by the drug combination versus doxorubicin alone.

### EXAMPLE 4

In this example, the cellular response to non-anthracycline agents, such as cisplatin and vinblastine, was investigated.

The experiment was carried out as described above and data represent the mean±s.e. of six determinations. The response to cisplatin was studied in SKN-SH wild type, SKN-SH wild type plus cathepsin L inhibition, SKN-SH doxorubicin cells, and SKN-SH doxorubicin resistant cells plus cathepsin L inhibition. These cells were found to have IC₅₀ values (to cisplatin LogM) of about −7.25, −6.75, 4.50, and −5.50, respectively. Interestingly, cellular response to cisplatin was not significantly affected by cathepsin inhibition in both doxorubicin sensitive and resistant cells. The data indicates that doxorubicin resistant SKN-SH/R cells were not resistant to cisplatin. This represents an additional argument in favor of the observation made earlier in Example 3, indicating that cathepsin L inhibition enhances cytotoxic drug response only in drug resistant cells. In this case, since there was no resistance to cisplatin, no resistance reversal should be expected.

In response to vinblastine (LogM), SKN-SH wild type, SKN-SH wild type plus cathepsin L inhibition, SKN-SH doxorubicin cells, and SKN-SH doxorubicin resistant cells plus cathepsin L inhibition had IC₅₀ values of about −7.0, −7.25, −5.25, and −6.25, respectively. In contrast to the results with cisplatin, the doxorubicin cells were also resistant to vinblastine. More importantly, cathepsin L inhibition reversed this resistance. Overall, the data suggest that reversal of drug resistance upon inhibition of cathepsin L function is valid for more than one chemotherapeutic agent and various cancer types.

### EXAMPLE 5

This example illustrates that cathepsin L inhibition results in acceleration of doxorubicin-induced expression of p21/WAFI and activation of caspase-3.

Doxorubicin resistant human neuroblastoma cells (SKN-SH/R) were subjected, in 25 cm² flasks, to treatment with a cathepsin L inhibitor and doxorubicin each, alone or in combination. After 24 hours of incubation, culture medium was removed and the cells washed twice with PBS. Proteins were solubilized with 150 μl of lysis buffer (50 mM HEPES pH 7.4, 150 mM NaCl, 100 mM NaF, 1 mM MgCl₂, 1.5 mM EGTA, 10% glycerol, 1% Triton X100, 1 μg/ml leupeptin, 1 mM phenyl-methyl-sulfonyl-fluoride). Equal quantities of protein were separated by electrophoresis on a 12% SDS-PAGE gel and transferred to Immobilon-P membranes (Millipore, Bedford, Mass.). P21/WAF1 and cleaved (active) caspase-3 were detected by reaction with specific primary antibodies (P21/WAF1 primary antibody from Santa Cruz Biotechnologies, Santa Cruz, Calif.; and caspase-3 primary antibody from Cell Signaling Technology, Inc., Beverly, Mass.) after one hour of incubation at room temperature in PBS (pH7.4). This was followed by incubation of the membrane for 1 hour at room temperature with presence of secondary anti-Rabbit antibody linked to horseradish peroxidase (Bio-Rad Laboratories, Hercules, Calif.) (1/1000 in PBS). Reactive bands were detected by chemiluminescence.

The combination of the cathepsin L inhibitor (10 μM) and doxorubicin at the sub-lethal concentration of 10⁻⁷M, enhanced expression of p21/WAF1 as indicated by the darkened band of the Western blot. Doxorubicin concentrations above and below 10⁻⁷ did not result in enhanced expression, or the expression was minimal. Interestingly, treatment of drug resistant cells with a higher doxorubicin concentration (10⁻⁶M) in the presence of cathepsin L inhibitor (10 μM) resulted in decreased expression of p21/WAF1 that was associated with increased caspase-3 activation, suggesting a switch of cell toxic response from proliferation arrest to apoptosis. The present findings indicate that cathepsin L inhibition accelerates both drug-induced proliferation arrest and cell death.

### EXAMPLE 6

This example illustrates that doxorubicin-resistant cells treated with siRNA directed to cathepsin L become more sensitive to doxorubicin.

The human cathepsin L siRNA was designed in our laboratory by studying a cDNA sequence of the human cathepsin L gene (SEQ ID NO: 2). We selected a segment defined by nucleotide numbers 91-111, sent that sequence to a contract laboratory (Dharmacon, Lafayette, Colo.), which synthesized an siRNA molecule based on the aforementioned segment. The segment of the human cathepsin L cDNA we used was: AAGTGGAAGGCGATGCACAAC (91-111) (SEQ ID NO: 2). On the day before transfection, 3×10⁵ drug resistant osteosarcoma cells (Saos2-R) were seeded in 6-well plates and grown in 2.5 ml of DMEM supplemented with 10% FBS. After 24 hours in culture, 25 μl of 20 μM stock solution of siRNA duplexes were transfected into cells with GeneSilencer™ SiRNA Transfection Reagent Kit (Gene Therapy Systems, Inc., San Diego, Calif.). Briefly, adherent cells were transfected in 6 well plates. Cells were about 70% confluent. In one tube, 5 μl of GeneSilencer™ was mixed with 25 μl of serum free medium. In a second tube, 25 μl of siRNA diluent was mixed with 15 g of serum free medium and 20 μl of siRNA (100 nM). After incubation of tubes 1 and 2, at room temperature for 5 min, they were mixed together into tube 3 and incubated for an additional 5 min. The content of tube 3 was added to the cells in the 6 well plates and incubated for 2 days before the addition of the drug. After 48 hours of incubation, doxorubicin (10⁻⁵ M) was added and maintained in culture for an additional 48 hours before analysis. The cells were counted and protein lysates were used to detect cathepsin L expression by Western blot as described above.

Western blot analysis showed that cathepsin L expression was completely diminished in cells transfected with the above described siRNA as well as in cells transfected with siRNA and treated with doxorubicin. The cell number, in response to the treatment protocol, was reduced from 1.6e+6 AU to 8.0e+5, 2.0e+5, and 1.5e+5 in cells treated with siRNA transfection alone, cells treated with doxorubicin and cathepsin L inhibition, and cells transfected with siRNA and treated with doxorubicin respectively. While cell treatment with doxorubicin or siRNA alone inhibited proliferation to certain extent, the combination of both inhibited cell proliferation by almost 90%. Similar results were obtained when doxorubicin was combined with the chemical cathepsin inhibitor. The data demonstrate that transfection of Saos2/R cells with cathepsin L siRNA results in almost complete suppression of the enzyme expression. The data are in support of the previous findings obtained with the combination of doxorubicin with the chemical inhibitor of cathepsin L and constitute an independent method to demonstrate the specificity of cathepsin L inhibition and its role in reversing drug resistance in cancer cells.

### EXAMPLE 7

This example sets forth data derived from testing additional cell lines.

The following additional cell lines were tested: breast cancer (MCF-7/doxR) (MCF: ATCC Cat. No. HTB-22) and ovarian cancer (A2780/CR; European collection of cell culture cat# 931112519, Salisbury, UK). Cells were treated with doxorubicin (10⁻⁵M) and/or a cathepsin L inhibitor (25 μM) for eight days. Viable cell number was counted and normalized to 100% of non-treated cells. From a control value of about 100%, doxorubicin reduced the percentage of viable MCF-7/doxR cells to about 50%, cathepsin inhibition alone reduced viable cells to about 90% and a combination of doxorubicin and cathepsin inhibition reduced viable cells to less than about 10%. Similarly, the percentage of viable A2780/CR cells was reduced from about 100% to about 70% with doxorubicin, about 90% with cathepsin L inhibition, and about 5% with doxorubicin and cathepsin L inhibition.

These results indicate that breast cancer and ovarian cancer cells are also susceptible to reversal of resistance to doxorubicin by inhibition of cathepsin L.

### EXAMPLE 8

This example illustrates that cathepsin L inhibition can prevent development of drug resistance in a cancer cell.

As discussed above, inhibition of cathepsin L enhanced drug response only in drug-resistant cells. Another aspect of the present invention was to determine whether treatment of drug-sensitive cancer cells with this drug combination prevents them from becoming drug resistant. Drug-sensitive cells SKN-SH and Saos2 were subjected to treatment 10⁻⁸ M doxorubicin with or without a cathepsin L inhibitor (Napsule-Ile-Tryp; Biomol, Plymouth Meeting, Pa.) at 10 μM. After 4 days in culture, the surviving cells were subjected to the same treatment for an additional four days. The cells were then subjected to two subsequent treatments for four days with 2.5×10⁻⁸ M doxorubicin with or without the cathepsin L inhibitor at 10 μM. The surviving cells were then treated with doxorubicin 5×10⁻⁸ M doxorubicin with or without cathepsin L (10 μM) for four days. At the end of each incubation period, viable cells were counted.

The results are as follows with and data representing the average±s.e. of three determinations. At each concentration, there is no change in the percentage of viable cells treated with doxorubicin alone. However, cells treated with doxorubicin and a cathepsin L inhibitor had a reduction in viable cells from about 80% (Dox 10⁻⁹ M) to almost 0 (2 treatments of Dox 2.5×10⁻⁸ M). Treatment of Saos2 cells with doxorubicin alone had no affect on the percentage of viable cells, whereas treatment with doxorubicin and cathepsin L inhibition resulted in a decrease of viable cells from about 90% (Dox 10⁻⁹ M) to about 0 (two treatments of Dox 2.5×10⁻⁸ M). As shown, both cell types have the ability to develop resistance to doxorubicin and resistant cells can be generated after only few passages in the presence of increasing drug concentrations. However, when the cathepsin L inhibitor was added to the culture, both cell lines lost the ability to become doxorubicin resistant. The data indicates that cathepsin L inhibition prevents development of drug resistance.

### EXAMPLE 9

This example illustrates the response of drug-resistant cells to cathepsin inhibition and treatment with additional chemotherapeutics.

SHN-SH cells were prepared in the manner described above. The SKN-SH doxorubicin-resistant cells were treated with chemotherapeutic agents alone (at concentrations of 10⁻⁷ to 10⁻⁴ M) or in the presence of a cathepsin L inhibitor. Viable cells were counted after 72 hours of incubation with the melphalan, etoposide, mitoxantrone, cyclophosphamide, and tamoxifen.

The number of viable cells was reduced from about 100% to about 60%, about 30%, and about 0 at melphalan concentrations of 10⁻⁸M, 10⁻⁷M, and 10⁻⁶ M, respectively. Treatment with melphalan and cathepsin L inhibition reduced the viable cells from about 100% to about 40%, about 10% and about 0 at melphalan concentrations of 10⁻⁸M, 10⁻⁷M, and 10⁻⁶M, respectively.

The number of viable cells was reduced from about 100% to about 95%, about 90%, about 70% and about 10% at etoposide concentrations of 10⁻⁷M, 10⁻⁶M, 10⁻⁵M, and 10⁻⁴ M, respectively. Treatment with etoposide and cathepsin L inhibition reduced the number of viable cells from about 100% to about 70%, about 65%, about 30% and about 5% at etoposide concentrations of 10⁻⁷M, 10⁻⁶M, 10⁻⁵M, and 10⁻⁴ M, respectively.

The number of viable cells was reduced from about 100% to about 40%, about 10% and about 5% at mitoxantrone concentrations of 10⁻⁸M, 10⁻⁷M, and 10⁻⁶ M, respectively. Treatment with mitoxantrone and cathepsin L inhibition reduced the number of viable cells from about 100% to about 10%, about 5% and about 5% at mitoxantrone concentrations of 10⁻⁸M, 10⁻⁷M, and 10⁻⁶ M, respectively.

The number of viable cells was reduced from about 100% to about 95%, about 80%, about 70%, and about 70% at cyclophosphamide concentrations of 10⁻⁷M, 10⁻⁶M, 10⁻⁵M, and 10⁻⁴ M, respectively. Treatment with cyclophosphamide and cathepsin L inhibition reduced the number of viable cells from about 100% to about 70%, about 65%, about 60% and about 30% at cyclophospamide concentrations of 10⁻⁷M, 10⁻⁶M, 10⁻⁵M, and 10⁻⁴ M, respectively.

That the number of viable cells was reduced from about 100% to about 75%, about 60%, about 60%, and about 0 at tamoxifen concentrations of 10⁻⁸M, 10⁻⁷M, 10⁻⁶ M, and 10⁻⁵M, respectively. Treatment with tamoxifen and cathepsin L inhibition reduced the number of viable cells from about 100% to about 40%, about 25%, about 25%, and about 0 at tamoxifen concentrations of 10⁻⁸M, 10⁻⁷M, 10⁻⁶ M, and 10⁻⁵M, respectively.

The data indicate that inhibition of cathepsin L is effective to reverse resistance of cells to other chemotherapeutic agents.

### EXAMPLE 10

This example illustrates the role of cathepsin L inhibition in senescence-mediated drug resistance reversal.

Antisense and siRNA oligonucleotides against cathepsin L are utilized to determine whether loss of the enzyme's function alters cell sensitivity to doxorubicin. Over expression of cathepsin L in cancer cells is also carried out. Putative relationships between cathepsin L and P-glycoprotein expression are also investigated.

We are using antisense complementary to any segment along the cathepsin L gene that inhibits cathepsin L expression. One example is an antisense oligonucleotide (CAG CAA GGA TGA GTG TAG GAT TCA T; SEQ ID NO: 3) (Gene Tools, Philomath, Oreg.), designed from the human cathepsin L gene, is used. Delivery of oligonucleotides is performed on cells seeded at 5×10⁵ cell/ml in 6 well plates and incubated for 24 hours. Oligonucleotides are added at 10 μM final concentration and the incubated cells are scraped to allow opening of holes into the plasma membrane and final entry of antisense molecules inside the cells. In one experiment, cells are transferred to 25 cm² flasks and incubated in culture medium for periods of time ranging from 8-96 hours. The cells are then lysed and expression of cathepsin L is determined by Western blot using a specific antibody that is labeled using a standard fluorescent tag (e.g., fluoroscene or rhodamine). In another set of experiments, cells are transferred to a 96 well plate and incubated for an additional 8 hours, then challenged with increasing doxorubicin concentration varying from 10⁻⁹ to 10⁻⁵ M. After 96 hours of incubation, MTT (3-(4,5-dimethyl-2-thiazolyl) 2,5-diphenyl tetrazolium bromide) is added and cell viability is determined. Drug toxicity is compared to non-transfected cells. Cathepsin L activity is measured in vitro and in intact cells by a CV-Cathepsin L Detection Kit (Biomol, Plymouth Meeting, Pa.) utilizing the fluorphore Cresyl Violet linked to phenylalanine-arginine (CV-(FR)2) as a substrate for cathepsin L.

Fragments of siRNA are generated from human cathepsin L cDNA (Invitrogen, Carlsbad, Calif.) by using the Dicer siRNA Generation kit (Gene Therapy Systems, San Diego, Calif.). Cathepsin L plasmid is amplified in E. coli then extracted using Qiagen™ extraction kit (Valencia, Calif.). A cathepsin L fragment of approximately 500 to 100 bp is generated by restriction enzymes. The fragment is used to generate dsRNA and siRNAs as follows: a T7 promoter (TAATACGACTCACTATAGGGAGA) (SEQ ID NO: 4) is added at both ends of a cathepsin L DNA fragment by using PCR so that it can be used as a template for in vitro transcription by the Turboscript™ T7 transcription kit. The 5′ primer, 5′-GCG-TAATACGACTCACTATAGGGAGAAGA-NNNNNN-3′ [SEQ ID NO:5], and the identical 3′ primer, 5′-GCG-TAATACGACTCACTATAGGGAGAAGA-NNNNNN-3′ [SEQ ID NO: 5], are incubated with 50 ng of DNA template in the reaction mix containing 10 μl 10×PCR buffer, 1 μl of 10 mM each dNTP, 1 μl of each primer (1 μg/μl), x μl of DNA polymerase (depending on supplier) and 86-x μl ddH20. The PCR program is 94° C. for three minutes, followed by 35 cycles of (94° C. for 30 seconds, 58° C. for 30 seconds, 68° C. for one min/kb) and 68 for five minutes. The PCR product is then used to generate dsRNA by incubating in 20 μl total volume, 8 μl of NTP mix, 2 μl of T7 reaction buffer, 1 μg PCR template DNA and 2 μl T7 enzyme mix. After two to four hours incubation at 37° C., dsRNA produced is checked on 1% agarose gel. siRNAs are generated by using recombinant dicer enzyme. Cell transfection with siRNA is carried out by the same method described above for antisense nucleotides.

Expression of cathepsin L in transfected and non-transfected cells is determined by Western blot. Cathepsin L is identified by reaction with specific primary and secondary antibodies linked to horseradish peroxidase. Reactive bands are detected by chemiluminescence.

Both drug sensitive and drug resistant intact cells are seeded at 10⁴ to 10⁵ cells onto a sterile coverslip in a 24 well plate in DMEM containing 10% FBS. When cells are 80% confluent, CV-(FR)2 (a substrate for cathepsin L provided by CV-Cathepsin L Detection Kit; Biomol, Plymouth Meeting, Pa.) is added (1/25 dilution) and after 30 min incubation at 37° C. the media is removed and cells are washed three times with PBS. Photographs are taken immediately with confocal microscope (excitation 550 nm, emission 610 nm). The sub-cellular localization of active cathepsin L is also compared between drug resistant and drug sensitive cells.

In vitro, cells are grown in 25 cm² flasks until 80% confluency are washed with ice cold PBS and lysed in 100 mM sodium acetate pH 5, 1 mM EDTA, and 1% triton X-100. After protein is assayed, 20 μg of proteins are incubated with or without cathepsin L inhibitor (10 μM) in a 96 well plate for 15 min at room temperature in 100 μl of reaction buffer (100 mM sodium acetate pH 5, 1 mM EDTA, and 4 mM dithiothreitol). 100 μl of substrate CV-(FR)2 is added and incubated for 30 min at room temperature. Fluorescence is measured in a plate reader (Victor Multilabel Counter, Perkin Elmer) at 550 nm excitation and 610 nm emission wave lengths.

Cytotoxic drug activity is quantitatively determined by colorimetric assay using 3-(4,5-dimethyl-2-thiazoyl)2,5-diphenyl tetrazolium bromide (MTT). Cells are seeded at 10⁴ cells/well and 96 well plates and maintained in culture for 24 hours at 37° C. in DMEM supplemented with 10% FBS. Drugs are added to designated wells and cells are incubated for 96 hours, following which MTT (10 μl of 5 mg/ml solution) will be added to each (100 μl) and incubation for 4 hours at 37° C. The cells are solubilized by incubation with μl of HCL 0.5N in isopropanol for 15 hours at 37° C. The optical density of this solution is measured at 570 nm and the percentage of viable cells estimated by comparison with untreated control cells.

A plasmid containing full-length human cathepsin L cDNA (ATCC) is transfected into drug sensitive and resistant SKN-SH cells as follows: the vector with or without the gene is introduced into cells using the cationic liposome system DOTAP (Boeringer Mannheim, Indianapolis, Ind.), according to the manufacturer's procedure. Putative transfectants are grown in a selection medium containing the antibiotic G418. Overexpression of cathepsin L in individual clones is confirmed by Western blot. Cellular response to doxorubicin is measured and compared between transfected and non-transfected cells. The relationship between overexpression of cathepsin L and expression of P-glycoprotein is studied by comparing expression of these two molecules using Western blot. Transfection where cathepsin L is down-regulated results in increased sensitivity to chemotherapeutic agents.

### EXAMPLE 11

This example illustrates the mechanism(s) by which inhibition of cathepsin L facilitates senescence and reversal of drug resistance.

SKN-SH/R cells are incubated with doxorubicin (10⁻⁹ to 10⁻⁷ M for 24 hours) in the presence or absence of cathepsin L inhibitor (10 μM). The cells are harvested in trypsin and centrifuged at 1,000×g for 5 min at 4° C. The cellular pellet is immediately fixed in 2.5% glutaraldehyde, post-fixed with 2% osmium tetroxide and processed for electron microscopy using conventional techniques. Ultra-thin sections stained with lead citrate and uranyl-acetate are examined with a Zeiss-10A electron microscope (Carl Zeiss Inc., Oberkochen, Germany). The presence of electron dense bodies (Lipofuscin) is compared in drug resistant and drug sensitive cells incubated with inhibitors for cathepsin L, cathepsin B, and the proteasome.

Cells are seeded on coverslips and incubated in DMEM containing 10% FBS for 24 hours. Lyso-Tracker™ (Molecular Probes, Eugene, Oreg.) or Acridine Orange (Molecular Probes, Eugene, Oreg.) is added in the absence or presence of cysteine protease inhibitors and incubated for 30 min at 37° C. The cells are washed three times with cold PBS and the intracellular localization of these dyes is examined by fluorescence microscopy (excitation 480 nm/emission 560 nm) and photographs are taken.

Expression of p21/WAF1 at the message level in response to doxorubicin and cathepsin L inhibitor is determined by quantitative RT-PCR:

Total RNA is isolated from drug resistant SKN-SH/R cells incubated with cathepsin L inhibitor (10 μM) in the absence and/or in the presence of doxorubicin (10⁻⁷ M) for 24 hours. The media is removed and the cells lysed with the QIAshredder™ (Qiagen, Valencia, Calif.). Total RNA is obtained by the RNeasy™kit (Qiagen, Valencia, Calif.) as recommended by the manufacturer. cDNA synthesis is performed with Omniscript reverse transcriptase (Qiagen, Valencia, Calif.) and random primer pd(N)₆ (Roche Diagnostics, Indianapolis, Ind.) and oligo(dT)₁₆ (MWG Biotech, Highpoint, N.C.). cDNA (50 ng) is incubated with SYBR Green PCR buffer, nucleotides, AmpliTaq Gold DNA polymerase (PE Biosystems, Foster City, Calif.) and the primers for the p21/WAF1 gene (forward 5′ CTG CCC AAG GCT TAC CTT CC-3′ (SEQ ID NO: 6), reverse 5′-CAG GTC CACATGGTCTTCCT-3′ (SEQ ID NO: 10)) each at 0.2 μM final concentration. For semiquantitative analysis, 40 cycles (denaturation: 94° C., 1 min; annealing and elongation: 60° C., 1 min) are performed in a Perkin Elmer GeneAmp PCR System 9600 equipped with a GeneAmp 5700 Sequence Detection System for quantification of PCR products. Agarose gel electrophoresis is used to verify the quality of PCR products. The data obtained is compared to standard curves obtained with plasmids containing authentic cDNAs of the p21/WAFT gene. Finally, the values are normalized to the results of GAPDH-RT-PCR.

Expression of p21/WAF1 at the message level in response to doxorubicin and cathepsin L inhibitor is determined by Northern Blot.

Drug resistant SKN-SH/R cells are incubated with cathepsin L inhibitor (10 μM) in the absence and/or in the presence of doxorubicin (10⁻⁷M) for 24 hours. Total RNA is extracted using an RNeasy™ mini-kit (Qiagen, Valencia, Calif.), run on a formaldehyde-containing 1% agarose gel, and transferred onto Hybond-N nylon filters (Amersham Biosciences, Piscataway, N.J.). The p21 probe is obtained by digesting the pET/p21/His plasmid, containing the human p21 cDNA, with BamHI and NcoI to obtain the full-length p21 cDNA. The probe is labeled with [³²P]dCTP (3000 Ci/mmol) using a random primer labeling kit (Amersham Biosciences). Filters are prehybridized for 2 h at 42° C. in 50% formamide, 5×SSC, 0.5% SDS, 0.2% polyvinylpyrrolidone, 0.2% Ficoll, 50 mM sodium pyrophosphate, pH 6.5, 1% glycine, and 500 μg/ml ssDNA. Hybridization is conducted for 15 h at 42° C. in 50% formamide, 5× sodium chloride-sodium citrate (SSC), 0.5% SDS, 0.04% polyvinylpyrrolidone, 0.04% Ficoll, 20 mM sodium pyrophosphate, pH 6.5, 10% dextran sulfate, and 100 μg/ml ssDNA. Filters are washed for 30 min with 2×SSC and 0.1% SDS at room temperature, followed by 60 min with 0.1×SSC and 0.1% SDS at 60° C. The glyceraldehyde-3-phosphate dehydrogenase probe is used to control the amount of loaded RNA. Expression of p21/WAF1 is compared between treated and untreated samples.

Study of the p53-Mitochondrial Pathway:

The SKN-SH/R cells are treated with cathepsin L inhibitor in the presence or the absence of doxorubicin at 10⁻⁷M for 24 hours. Expression and phosphorylation of p53, Fas expression and activation of caspase-9 are detected by western blot using specific antibodies.

Mitochondrial permeability transition is studied in cells seeded on coverslips and treated as above with cathepsin L inhibitor and doxorubicin. The cells are then washed three times with PBS and the mitochondrial transmembrane potential is measured by incubation with JC1 fluorophore (10 μg/ml; Cell Technology Inc., Minneapolis, Minn.) for 10 min at 37° C. The cells are washed three times with PBS, the coverslips placed on slides and cells are analyzed under fluorescence microscopy (excitation 485 nm/emission 530 nm).

To measure cytochrome c release, cells are harvested with trypsin and the cell suspension centrifuged at 1,000×g for 5 min at 4° C. After washing with ice cold PBS, mitochondria is isolated by resuspending the cells in five volumes of ice cold buffer (20 mM Hepes-KOH, pH 7.5, 10 mM KCl, 1.5 mM MgCl₂, 1 mM Sodium EDTA, 1 mM sodium EGTA, 1 mM dithiothreitol) containing 250 mM sucrose. Cells are lysed by 15-20 passages through a 25-gauge needle, and homogenate centrifuged at 1000×g for 5 min at 4° C. Supernatants are centrifuged at 10,000×g for 15 min at 4° C., and the resulting mitochondria pellets are re-suspended in 50 μl of lysis buffer. Cytochrome c is detected in the mitochondrial pellet and the corresponding supernatant (cytoplasm) by Western blot using a specific antibody.

Electron microscopy is utilized to identify possible alterations in lysosomal structure. Special emphasis is on the apparition of electron dense bodies in the cytoplasm following cathepsin L inhibition. These bodies are thought to accumulate non-degraded proteins which may cause an increase in lysosomal pH. Confocal microscopy experiments using Acridine Orange are conducted to confirm the increase in lysosomal pH as a result of cathepsin L inhibition.

Comparison of p21/WAF1 expression at the message level (by PCR or Northern Blot) and at the protein level in response to treatment with cathepsin L inhibitor and doxorubicin allows us to determine whether this drug combination induces p21/WAF1 expression or reduces its degradation. Since we have found that p2l/WAF1 is readily cleaved by cathepsin L in vitro, we believe that this cell cycle inhibitor is a physiological substrate for cathepsin L and that its cleavage is of relevance in explaining the survival function of this enzyme.

Increased p21/WAF1 amounts after cell treatment with doxorubicin and cathepsin L are due to increased mRNA expression of this molecule, and suggests that expression or function of the upstream regulator p53 is also enhanced. The function of p53, the Fas ligand and eventually the downstream mitochondrial pathway are also activated upon cell treatment with the drug combination. However, since caspase-3 activity was not enhanced, this suggests that either cathepsin L has a target in this pathway that is inactivated upon cathepsin L inhibition (cathepsin L has been shown to activate Bid, therefore its inhibition may inactivate the Bid pathway), or that the mitochondial pathway does not mediate cathepsin L action.

It is therefore intended that the foregoing detailed description be regarded as illustrative rather than limiting, and that it be understood that it is the following claims, including all equivalents, that are intended to define the spirit and scope of this invention.

1. A method for increasing sensitivity of a cancer cell to a cytotoxic agent comprising:
a) contacting a cancer cell having a resistance to a cytotoxic agent with a cathepsin inhibitor; and
b) contacting the cancer cell with a cytotoxic agent.

2. A method for preventing resistance of a cancer cell to a cytotoxic agent comprising:
a) contacting a cancer cell with a sub-cytotoxic concentration of a cathepsin inhibitor; and
b) contacting the cancer cell with a cytotoxic agent.

3. The method of claim 1 or claim 2, wherein said cathepsin inhibitor reduces the enzymatic activity of the cathepsin.

4. The method of claim 1 or claim 2, wherein said cathepsin inhibitor reduces expression of the cathepsin.

5. The method of claim 3, wherein the cathepsin inhibitor is a small molecule.

6. The method of claim 4, wherein the cathepsin inhibitor comprises an antisense molecule 10-30 nucleotides in length that specifically hybridizes to and inhibits expression of a nucleic acid encoding a cathepsin.

7. The method of claim 6, wherein the antisense molecule specifically hybridizes to and inhibits expression of a nucleic acid encoding cathepsin-L.

8. The method of claim 7, wherein in the antisense molecule specifically hybridizes to a nucleic acid sequence as set forth in SEQ ID NO:7.

9. The method of claim 8, wherein the antisense molecule comprises an RNA.

10. The method of claim 8 or claim 9, wherein the antisense molecule is double stranded.

11. The method any of claims 1-10, wherein the cathepsin inhibitor inhibits cathepsin L.

12. The method claim 11, wherein the cathepsin inhibitor inhibits cathepsin L preferentially as compared to cathepsin S or cathepsin K.

13. The method any of claims 1-12, wherein the cancer cell is selected from the group consisting of a neuroblastoma cell, an osteosarcoma cell, a leukemia cell, a breast cancer cell, and an ovarian cancer cell.

14. The method any of claims 1-13, wherein the cancer cell is present in a subject.

15. The method of claim 14, wherein step a) comprises administering a dose of the cathepsin inhibitor to the subject.

16. The method of claim 15, wherein said dose of the cathepsin inhibitor comprises multiple administrations of the cathepsin inhibitor to the subject.

17. The method of claim 14 or claim 15, wherein step b) comprises administering a dose of the cytotoxic agent to the subject.

18. The method of claim 17, wherein said dose of the cytotoxic agent comprises multiple administrations of the cytotoxic agent to the subject.

19. The method of claim 17 or claim 18, wherein the cytotoxic agent is administered prior to administration of the cathepsin inhibitor.

20. The method of any one of claims 17-19, wherein the cytotoxic agent is administered after administration of the cathepsin inhibitor.

21. The method of any one of claims 17-20, wherein the cytotoxic agent is administered at the same time as the cathepsin inhibitor.

22. The method of any of claims 15-21, wherein the cathepsin inhibitor is administered by direct injection.

23. The method of any of claims 1-22, wherein the cytotoxic agent is nonmetal-based agent.

24. The method of claim 23, wherein the nonmetal-based agent is selected from the group consisting of doxorubicin, anthracycline, vinblastine, taxol, mitoxantrone, melphalan, etoposide, cyclophosphamide, and tamoxifen.

25. The method of any of claims 17-24, wherein the dose of cytotoxic agent is effective in preventing proliferation of cancer cells within a subject.

26. The method of claim 25, wherein the dose of cytotoxic agent is less than the dose effective in preventing proliferation of cancer cells within a subject in the absence of the cathepsin inhibitor.

27. A composition for performing the method of any one of claims 1-26, said composition comprising a cathepsin inhibitor and a cytotoxic agent.

28. Use of a cathepsin inhibitor in the preparation of a medicament for preventing resistance of a cancer cell to a cytotoxic agent.

29. The Use of claim 28, wherein said medicament further comprises a cytotoxic agent.


SEQUENCE LISTING (10 sequences)

SEQ ID NO: 1 - RNA, 21 nt, Homo sapiens
uucaccuuccgcuacguguug

SEQ ID NO: 2 - DNA, 21 bp, Homo sapiens
aagtggaaggcgatgcacaac

SEQ ID NO: 3 - DNA, 25 bp, Homo sapiens
cagcaaggatgagtgtaggattcat

SEQ ID NO: 4 - DNA, 23 bp, Homo sapiens
taatacgactcactatagggaga

SEQ ID NO: 5 - DNA, 35 bp, Homo sapiens
  misc_feature (30)..(35): n is a, c, g, or t
gcgtaatacgactcactatagggagaagannnnnn

SEQ ID NO: 6 - DNA, 20 bp, Homo sapiens
ctgcccaaggcttaccttcc

SEQ ID NO: 7 - DNA, 1002 bp, Homo sapiens
atgaatcctacactcatccttgctgccttttgcctgggaattgcctcagctactctaacatttgatcacagtttagaggcacagtggaccaagtggaaggcgatgcacaacagattatacggcatgaatgaagaaggatggaggagagcagtgtgggagaagaacatgaagatgattgaactgcacaatcaggaatacagggaagggaaacacagcttcacaatggccatgaacgcctttggagacatgaccagtgaagaattcaggcaggtgatgaatggctttcaaaaccgtaagcccaggaaggggaaagtgttccaggaacctctgttttatgaggcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtgagcagaatctggtagactgctctgggcctcaaggcaatgaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtttaa

SEQ ID NO: 8 - DNA, 987 bp, Homo sapiens
atgtgggggctcaaggttctgctgctacctgtggtgagccttgctctgtaccctgaggagatactggacacccactgggagctatggaagaagacccacaggaagcaatataacaacaaggtggatgaaatctctcggcgtttaatttgggaaaaaaacctgaagtatatttccatccataaccttgaggcttctcttggtgtccatacatatgaactggctatgaaccacctgggggacatgaccagtgaagaggtggttcagaagatgactggactcaaagtacccctgtctcattcccgcagtaatgacaccctttatatcccagaatgggaaggtagagccccagactctgtcgactatcgaaagaaaggatatgttactcctgtcaaaaatcagggtcagtgtggttcctgttgggcttttagctctgtgggtgccctggagggccaactcaagaagaaaactggcaaactcttaaatctgagtccccagaacctagtggattgtgtgtctgagaatgatggctgtggagggggctacatgaccaatgccttccaatatgtgcagaagaaccggggtattgactctgaagatgcctacccatatgtgggacaggaagagagttgtatgtacaacccaacaggcaaggcagctaaatgcagagggtacagagagatccccgaggggaatgagaaagccctgaagagggcagtggcccgagtgggacctgtctctgtggccattgatgcaagcctgacctccttccagttttacagcaaaggtgtgtattatgatgaaagctgcaatagcgataatctgaaccatgcggttttggcagtgggatatggaatccagaagggaaacaagcactggataattaaaaacagctggggagaaaactggggaaacaaaggatatatcctcatggctcgaaataagaacaacgcctgtggcattgccaacctggccagcttccccaagatg

SEQ ID NO: 9 - DNA, 996 bp, Homo sapiens
atgaaacggctggtttgtgtgctcttggtgtgctcctctgcagtggcacagttgcataaagatcctaccctggatcaccactggcatctctggaagaaaacctatggcaaacaatacaaggaaaagaatgaagaagcagtacgacgtctcatctgggaaaagaatctaaagtttgtgatgcttcacaacctggagcattcaatgggaatgcactcatacgatctgggcatgaaccacctgggagacatgaccagtgaagaagtgatgtctttgatgagttccctgagagttcccagccagtggcagagaaatatcacatataagtcaaaccctaattggatattgcctgattctgtggactggagagagaaagggtgtgttactgaagtgaaatatcaaggttcttgtggtgcttgctgggctttcagtgctgtgggggccctggaagcacagctgaagctgaaaacaggaaagctggtgtctctcagtgcccagaacctggtggattgctcaactgaaaaatatggaaacaaaggctgcaatggtggcttcatgacaacggctttccagtacatcattgataacaagggcatcgactcagacgcttcctatccctacaaagccatggatcagaaatgtcaatatgactcaaaatatcgtgctgccacatgttcaaagtacactgaacttccttatggcagagaagatgtcctgaaagaagctgtggccaataaaggcccagtgtctgttggtgtagatgcgcgtcatccttctttcttcctctacagaagtggtgtctactatgaaccatcctgtactcagaatgtgaatcatggtgtacttgtggttggctatggtgatcttaatgggaaagaatactggcttgtgaaaaacagctggggccacaactttggtgaagaaggatatattcggatggcaagaaataaaggaaatcattgtgggattgctagctttccctcttacccagaaatctag

SEQ ID NO: 10 - DNA, 20 bp, Artificial Sequence
  Synthetic oligonucleotide
caggtccacatggtcttcct